SEQUENCE LISTING (10 sequences)

SEQ ID NO: 1 - DNA, 1026 bp, Homo sapiens
aaaggacagatattgcagaagagagaaggtataactgggaccaaaagccttgagaaggaaagagacatggagcaaatcattcacagtaacagcagacagcagagaagagacacatggttgtacagaggcacctcctttgggtctttactcaaatgccccattatcagtgagaacttctctgactgctgttcttcagcagagggtattccttatcccctttcttgctttatgtgttttctccataacatatgtgcatatccataacacacacatgcatcacctagagcattatatatgccacagtgacatgttttgctgatttctcaattgactcccccattggaatgaacgtaagcttgaggaagacgttttgtcctgttctgtagcatctagaacagcgcctggcacatagtaggtactcaataaatgccagctgcatgaggaaatgaatgagctgtgtgggggatgtacttgagtgaactctaaagtcagagtggtgttgagagaaaaatgcttgaaatccagatgttggaaggtgacacagagtagtagcctggtgagaacagttagatcttaggggttcctactacagccctcccttccgcacctttttggctgtcaccatgatcaagctactgaatctctctgagacgcaaggaccgggatggcacaaagtgagtgctcaccaaagcttgactgtcctttcccatggcaatttacttcagcttgtttgatttcccctccccgactggactaggcacctattctctgtcttctctctttacagttggaaggagcaaaatgggacttttggctgaaagtgctgagctcctgcggtgggggctgaccgcaagccgcgccttctgtgcacctggtcggcccagctagctgcggacccggcggggaggggcggggcgggccaatcggcgctgccccagcagggctgcggctgcaggcagcagagcctcctagcccgtcggtgtctgcgcccatcgatccctttgtctatccccgaccatggcgaag

SEQ ID NO: 2 - DNA, 274 bp, Homo sapiens
caaggaccgggatggcacaaagtgagtgctcaccaaagcttgactgtcctttcccatggcaatttacttcagcttgtttgatttcccctccccgactggactaggcacctattctctgtcttctctctttacagttggaaggagcaaaatgggacttttggctgaaagtgctgagctcctgcggtgggggctgaccgcaagccgcgccttctgtgcacctggtcggcccagctagctgcggacccggcggggaggggcggggcgggccaatcgg

SEQ ID NO: 3 - DNA, 17 bp, Artificial Sequence
  PON1 SNP Primer 1
ccgattggcccgccccg

SEQ ID NO: 4 - DNA, 17 bp, Artificial Sequence
  PON1 SNP Primer 2
ccgattggcccgcccca

SEQ ID NO: 5 - DNA, 18 bp, Artificial Sequence
  PON1-107 consensus primer
caaggaccgggatggcac

SEQ ID NO: 6 - DNA, 19 bp, Artificial Sequence
  Forward control primer coding for a fragment of the HLA-DRB3 gene
tgccaagtggagcacccaa

SEQ ID NO: 7 - DNA, 20 bp, Artificial Sequence
  Reverse control primer coding for a fragment of the HLA-DRB3 gene
gcatcttgctctgtgcagat

SEQ ID NO: 8 - DNA, 21 bp, Artificial Sequence
  Reverse allele-specific primer for the PON1 M54L polymorphism
cagaaactggctctgaagaca

SEQ ID NO: 9 - DNA, 21 bp, Artificial Sequence
  Reverse allele-specific primer for the PON1 M54L polymorphism
cagaaactggctctgaagact

SEQ ID NO: 10 - DNA, 20 bp, Artificial Sequence
  M54L Consensus primer
aagtgggcatgggtatacag

(19) United States
(12) Patent Application Publication
(10) Pub. No.: US 2008/0241824 A1
(43) Pub. Date: Oct. 2, 2008

(54) Mutation Associated With Lacunar Strokes

(75) Inventors: Jim Jannes, South Australia (AU); Monica Anne Hamilton-Bruce, South Australia (AU); Simon Koblar, South Australia (AU)

Correspondence Address: BIRCH STEWART KOLASCH & BIRCH, PO BOX 747, FALLS CHURCH, VA, 22040-0747, US

(73) Assignees: THE QUEEN ELIZABETH HOSPITAL RESEARCH FOUNDATION INC., Woodville South (AU); Adelaide Research & Innovation Pty Ltd., Adelaide (AU)

(21) Appl. No.: 10/589,735

(22) PCT Filed: Feb. 18, 2005
(86) PCT No.: PCT/AU05/00218

§ 371 Date: Jan. 22, 2007

(30) Foreign Application Priority Data
Feb. 18, 2004 (AU) 2004900790

(51) Int. Cl. C12Q 1/68; C07H 21/04

(52) U.S. Cl. 435/6; 536/24.33

(57) ABSTRACT

The present invention relates to a method of identifying a subject predisposed to lacunar stroke. The method includes the step of identifying in the subject the presence of a thymine to cytosine mutation at position -107 in both alleles of the paraoxonase 1 locus.

### FIELD OF THE INVENTION

The present invention relates to methods of identifying a subject having a predisposition to lacunar stroke and to methods of identifying a subject having a predisposition to small vessel occlusion.

The present invention also relates to methods of treating a subject susceptible to a lacunar stroke and to methods of treating a subject suceptible to small vessel occlusion.

### BACKGROUND OF THE INVENTION

lschemic strokes result from the formation of an occlusive thrombus in one of the vessels of the brain. Ischemic stroke may be thrombotic or embolic in origin. In a thrombotic stroke, a blood clot develops in a vessel already narrowed by atherosclerosis. In an embolic stroke, a clot forms elsewhere in the body and travels through the circulatory system to the brain. Atherosclerosis is a major contributing factor in ischemic strokes.

One particular type of ischemic stroke is a lacunar stroke. A lacunar stroke is a small vessel occlusion involving the blockage of blood flow to a part of the brain supplied by one of the brain's smaller “penetrating arteries.” These small arteries branch from larger arteries on the underside of the brain and carry blood to the brain's deeper regions. Lacunar strokes are usually thrombotic or embolic in origin, although in some cases lacunar strokes can also arise from causes other than atherosclerosis, such as lipohyalinosis.

The oxidation of low-density lipoprotein (LDL) plays an important role in the pathogenesis of atherosclerosis. Oxidized LDL, when trapped in the sub-endothelial space, induces the release of monocyte-specific chemoattractants, adhesion molecules and colony stimulating factors from vascular endothelium.

Further oxidation leads to arterial wall cytotoxicity and uptake of oxidized LDL particles by macrophages. This sequence of events ultimately results in atherosclerotic plaque formation.

The oxidation of LDL is limited by high-density lipoprotein (HDL). This effect is mediated by paraoxonase 1 (PON1), an enzyme that is produced in the liver and circulates in the blood exclusively bound to HDL. PON-1 exerts an anti-atherogenic effect by hydrolysing sub-endothelial peroxides before they accumulate in LDL particles. In PON1 knockout mice, HDL loses its protective effect on LDL oxidation, and mice become susceptible to diet-induced atherosclerosis.

PON1 expression appears to be largely determined by genetic factors. Two single nucleotide polymorphisms (SNPs) have been identified in the PON1 coding region. In addition, a number of further SNPs have been identified in the PON1 regulatory region. One of these SNPs, -107 T/C, appears to be the main genetic contributor to PON1 expression. Possession of the T allele at position -107 has been shown to be associated with significantly lower PON1 levels and serum enzyme activity.

There is currently very little information regarding the importance that genetic factors play in the development of diseases and conditions associated with occlusive thrombosis, and in particular, the genetic factors that play a role in lacunar strokes and small vessel occlusion. As such, there is a need for methods of identifying subjects that may be susceptible to lacunar stroke and small vessel occlusion.

The present invention relates to methods of identifying subjects predisposed to lacunar stroke and small vessel occlusion by identifying the presence of a specific polymorphism in the upstream region of the PON1 gene. The present invention also relates to a method of identifying subjects predisposed to lacunar stroke and small vessel occlusion by identifying a serum activity of PON1 in the subject above a threshold value.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in any country. The discussion of the references states what their authors assert, and the applicant reserves the right to challenge the accuracy and pertinency of any of the documents cited herein.

### SUMMARY OF THE INVENTION

The present invention provides a method of identifying a subject predisposed to lacunar stroke, the method including the step of identifying in the subject the presence of a thymine to cytosine mutation at position -107 in both alleles of the paraoxonase 1 locus.

The present invention also provides a method of identifying a subject predisposed to small vessel occlusion, the method including the step of identifying in the subject the presence of a thymine to cytosine mutation at position -107 in both alleles of the paraoxonase 1 locus.

The present invention also provides a method of identifying a subject predisposed to developing a disease or condition associated with small vessel occlusion, the method including the step of identifying in the subject the presence of a thymine to cytosine mutation at position -107 in both alleles of the paraoxonase 1 locus.

The present invention also provides a method of determining the risk of lacunar stroke in a subject, the method including the step of determining in the subject the presence of a thymine to cytosine mutation at position -107 in one or both alleles of the paraoxonase 1 locus.

The present invention also provides a method of determining the risk of small vessel occlusion in a subject, the method including the step of determining in the subject the presence of a thymine to cytosine mutation at position -107 in one or both alleles of the paraoxonase 1 locus.

The present invention also provides a method of determining the risk of a subject developing a disease or condition associated with small vessel occlusion, the method including the step of determining in the subject the presence of a thymine to cytosine mutation at position -107 in one or both alleles of the paraoxonase 1 locus.

The present invention also provides a method of identifying a subject suitable for treatment with an agent that decreases the activity of paraoxonase 1, the method including the step of determining in the subject the presence of a thymine to cytosine mutation at position -107 in one or both alleles of the paraoxonase 1 locus.

The present invention also provides a method of treating a subject susceptible to small vessel occlusion or susceptible to a disease or condition associated with small vessel occlusion, the method including the step of administering to the subject an effective amount of an agent that decreases the activity of paraoxonase 1.

The present invention further provides a method of identifying a subject predisposed to lacunar stroke, the method including the step of identifying an arylesterase activity of paraoxonase 1 in serum from the subject of greater than 100 units per ml.

The present invention also provides a method of identifying a subject predisposed to small vessel occlusion, the method including the step of identifying an arylesterase activity of paraoxonase 1 in serum from the subject of greater than 100 units per ml.

The present invention also provides a method of identifying a subject predisposed to developing a disease or condition associated with small vessel occlusion, the method including the step of identifying an arylesterase activity of paraoxonase 1 in serum from the subject of greater than 100 units per ml.

The present invention also provides a method of determining the risk of lacunar stroke in a subject, the method including the step of identifying an arylesterase activity of paraoxonase 1 in serum from the subject of greater than 100 units per ml.

The present invention also provides a method of determining the risk of small vessel occlusion in a subject, the method including the step of identifying an arylesterase activity of paraoxonase 1 in serum from the subject of greater than 100 units per ml.

The present invention also provides a method of determining the risk of a subject developing a disease or condition associated with small vessel occlusion, the method including the step of identifying an arylesterase activity of paraoxonase 1 in serum from the subject of greater than 100 units per ml.

The present invention also provides a method of identifying a subject suitable for treatment with an agent that decreases the activity of paraoxonase 1, the method including the step of identifying an arylesterase activity of paraoxonase 1 in serum from the subject of greater than 100 units per ml.

The present invention also provides a method of identifying a subject suitable for intervention to prevent and/or treat a lacunar stroke, the method including the step of identifying in the subject the presence of a thymine to cytosine mutation at position -107 in both alleles of the paraoxonase 1 locus.

The present invention also provides a method of identifying a subject suitable for intervention to prevent and/or treat a small vessel occlusion, the method including the step of identifying in the subject the presence of a thymine to cytosine mutation at position -107 in both alleles of the paraoxonase 1 locus. The present invention also provides a method of identifying a subject suitable for intervention to prevent and/or treat a disease or condition associated with small vessel occlusion, the method including the step of identifying in the subject the presence of a thymine to cytosine mutation at position -107 in both alleles of the paraoxonase 1 locus.

The present invention also provides a method of identifying an agent for treating a subject susceptible to lacunar stroke, small vessel occlusion or a disease or condition associated with small vessel occlusion, the method including the steps of:
(a) exposing an agent to a cell expressing PON1, wherein the cell includes a mutation that results in overexpression of PON1 compared to a cell without the mutation;
(b) determining the level of expression or activity of PON1 from the cell; and
(c) identifying the agent as an agent capable of decreasing the expression and/or activity of PON1.

The present invention also provides a method of identifying an agent for treating a subject susceptible to lacunar stroke, small vessel occlusion or a disease or condition associated with small vessel occlusion, the method including the steps of:
(a) exposing an agent to a cell transformed with all or part of the PON1 locus, wherein the transformed locus includes a thymine to cytosine mutation at position -107 and the transformed locus regulates expression of a reporter gene;
(b) determining the level of expression of the reporter gene in the cell so exposed to the agent; and
(c) identifying the agent as an agent that decreases PON1 expression by the decrease in expression of the reporter gene.

The present invention also provides an isolated nucleic acid consisting of the sequence according to SEQ. ID No.3 or RNA equivalent thereof.

The present invention also provides an isolated nucleic acid with one or more base substitutions in the sequence according to SEQ ID No. 3, wherein the nucleic acid hybridises with the complement of SEQ ID No.3 under stringent hybridisation conditions and the stringent hybridisation conditions include hybridisation in 6×SSC at 42° C. and washing in 2SSC at 20° C.

The present invention also provides an isolated nucleic acid consisting of the sequence according to SEQ. ID No.4 or RNA equivalent thereof.

The present invention also provides an isolated nucleic acid with one or more base substitutions in the sequence according to SEQ ID No. 4, wherein the nucleic acid hybridises with the complement of SEQ ID No.4 under stringent hybridisation conditions and the stringent hybridisation conditions include hybridisation in 6×SSC at 42° C. and washing in 2×SSC at 20° C.

The present invention also provides an isolated nucleic acid consisting of the sequence according to SEQ. ID No.5 or RNA equivalent thereof.

The present invention also provides an isolated nucleic acid with one or more base substitutions in the sequence according to SEQ ID No. 5, wherein the nucleic acid hybridises with the complement of SEQ ID No. 5 under stringent hybridisation conditions and the stringent hybridisation conditions include hybridisation in 6×SSC at 42° C. and washing in 2×SSC at 20° C.

The present invention also provides an isolated nucleic acid with one or more base substitutions in the sequence according to SEQ ID No. 3, wherein the nucleic acid has at least 80% homology to SEQ. ID No.3 or RNA equivalent thereof.

The present invention also provides an isolated nucleic acid with one or more base substitutions in the sequence according to SEQ ID No. 4, wherein the nucleic acid has at least 80% homology to SEQ. ID No.4 or RNA equivalent thereof.

The present invention also provides an isolated nucleic acid with one or more base substitutions in the sequence according to SEQ ID No. 5, wherein the nucleic acid has at least 80% homology to SEQ. ID No.5 or RNA equivalent thereof.

The present invention also provides an isolated nucleic acid, the nucleic acid consisting of the sequence spanning nucleotides 654 to 927 of SEQ ID No. 1.

The present invention arises out of studies into the predisposition to lacunar strokes in humans. In particular, it has been found that a T to C mutation at position -107 in the regulatory region of the paraoxonase 1 (PON1) gene is an independent prognostic indicator for the risk of lacunar stroke.

This finding is contrary to expectation, as the posession of the C allele is associated with increased PON1 activity and an increase in PON1 activity would normally be expected to result in anti-athrogenic effect. Without being bound theory, it may be that excessive PON1 activity may result in overproduction and/or excess of toxic moieties, resulting in endothelial dysfunction and subsequent lipohyalinosis.

In addition, this finding also indicates that an increased serum activity of PON1 above a threshold value (100 units per ml) is likely to be an independent prognostic indicator for lacunar stroke.

Further, given that a lacunar stroke is a form of small vessel occlusion, this finding also indicates that the -107 T to C mutation is likely to be a prognostic indicator for the risk of a subject suffering a small vessel occlusion, or developing a disease or condition associated with small vessel occlusion.

Various terms that will be used throughout the specification have meanings that will be well understood by a skilled addressee. However, for ease of reference, some of these terms will now be defined.

The term “PON1 locus” as used throughout the specification is to be understood to mean any DNA associated with the coding region of the PON1 gene, any DNA coding for an untranslated region of a PON1 mRNA, any DNA coding for a PON1 pre-mRNA (eg intronic sequences) or any DNA associated with the regulation of PON1 expression, such as a promoter element, an enhancer element (proximal or distal), a binding site for a regulatory factor (eg a SP1 binding site) or any other DNA that has a role in expression of the PON1 locus, such as heterochromatic DNA.

The term “predisposed” as used throughout the specification in relation to lacunar stroke or small vessel occlusion is to be understood to mean the increased probability that a subject with a mutation will suffer a lacunar stroke or a small vessel occlusion, as compared to the probability that another subject not having the same mutation will suffer a lacunar stroke or a small vessel occlusion, under circumstances where other risk factors (eg atrial fibrillation, history of smoking) for having an lacunar stroke or a small vessel occlusion between the subjects are the same.

In this regard, it will also be understood that the term “predisposed” when used in relation to a disease or condition associated with small vessel occlusion is to be understood to mean the increased probability that a subject with a mutation will develop a disease or condition associated with small vessel occlusion, as compared to the probability that another subject not having the same mutation will develop a disease or condition associated with small vessel occlusion, under circumstances where other risk factors (eg atrial fibrillation, history of smoking) between the subjects are the same.

The term “mutation” as used throughout the specification is to be understood to mean any change in the normal DNA sequence. As will be appreciated a mutation will also be polymorphic. Examples of types of mutations include an insertion, deletion, frameshift, or base substitution.

The term “small vessel occlusion” as used throughout the specification is to be understood to mean the occlusion by thrombus of any blood vessel less than 800 micrometres in diameter. The definition includes pre-capillary arterioles, capillaries and post-capillary venules. Within the brain, small vessel occlusion may manifest clinically as lacunar stroke, thalamic infarction, white matter medullary infarction or generalized subcortical leukoariosis. The latter pathological entity underlies vascular dementia.

A “polymorphism” as used throughout the specification is to be understood to mean a difference in DNA sequence among individuals.

The terms “amplification” or “amplify” (or variants thereof) as used throughout the specification is to be understood to mean the production of additional copies of a nucleic acid sequence. For example, amplification may be achieved using polymerase chain reaction (PCR) technologies (as described in Dieffenbach, C. W. and G. S. Dveksler (1995) PCR Primer, a Laboratory Manual, Cold Spring Harbor Press, Plainview, N.Y.) or by other methods of amplification, such as rolling circle amplification on circular templates, such as described in Fire, A. and Xu, S-Q. (1995) Proc. Natl. Acad. Sci 92:4641-4645.

The term “nucleic acid” as used throughout the specification is to be understood to mean any polynucleotide or oligonucleotide, being composed of deoxyribonucleotides or ribonucleotides in either single- or double-stranded form, including known analogues of natural nucleotides that can function in a similar manner as naturally occurring nucleotides.

In this regard, the nucleic acid may be modified at the base moiety, sugar moiety, or phosphate backbone, and may include other appending groups to facilitate the function of the nucleic acid.

For example, the nucleic acid may include at least one modified base moiety such as 5-fluorouracil, 5-bromouracil, 5-chlorouracil, 5-iodouracil, hypoxanthine, xanthine, 4-acetylcytosine, 5-(carboxyliydroxylmethyl) uracil, 5-carboxymethylaminomethyl-2-thiou rid ine, 5-carboxymethylaminomethylu racil, dihydrouracil, beta-D-galactosylqueosine, inosine, N6-isopentenyladenine, 1- methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

The nucleic acid may also include at least one modified sugar moiety selected from the group including arabinose, 2-fluoroarabinose, xylulose, and hexose. In addition, the nucleic acid may include at least one modified phosphate backbone, such as a phosphorothioate, a phosphorodithioate, a phosphoramidothioate, a phosphoramidate, a phosphordiamidate, a methylphosphonate, an alkyl phosphotriester, and a formacetal or any analogue thereof.

The term “isolated nucleic acid” as used throughout the specification is to be understood to mean a nucleic acid which is substantially separated from other cellular components which naturally accompany the nucleic acid. The term includes a nucleic acid sequence which has been removed from its naturally occurring environment, and includes natural, cloned or recombinant DNAs, chemically synthesized nucleic acids, such as chemically synthesized oligonucleotides, any DNA or RNA biologically synthesized by a heterologous system, or any. other form of polynucleotide analogue.

In this regard, a nucleic acid isolated or derived from a subject is any nucleic acid that has as its ultimate source a nucleic acid obtained from a subject. For example, a nucleic acid isolated or derived from a subject may be a genomic clone of a specific region, a cDNA clone of a specific region, or a nucleic acid obtained by amplifying genomic DNA, mRNA or cloned DNA.

The term “hybridises” or “hybridisation” (or variants thereof) as used throughout the specification is to be understood to mean any process by which a strand of nucleic acid binds with a complementary strand through base pairing. Hybridisation may occur in solution, or between one nucleic acid sequence present in solution and another nucleic acid sequence immobilized on a solid support (e.g., membranes, filters, chips etc).

The term “stringent conditions” as used throughout the specification is to be understood to mean the conditions that allow complementary nucleic acids to bind to each other within a range from at or near the Tm (Tm is the melting temperature) to about 20° C. below Tm. Factors such as the length of the complementary regions, type and composition of the nucleic acids (DNA, RNA, base composition), and the concentration of the salts and other components (e.g. the presence or absence of formamide, dextran sulfate and/or polyethylene glycol) must all be considered, essentially as described in Current Protocols in Molecular Biology, John Wiley & Sons, N.Y. (1989).

The term “upstream region” in relation to the PON1 locus as used throughout the specification will be understood to mean those sequences normally within 10 kb of the start of the transcribed region of the PON1 locus (but which may also be more distant from the coding region), which affect the expression of the gene (including transcription of the gene, and translation, splicing, stability or the like of the messenger RNA).

In this regard, sequence coordinates used throughout the specification in relation to the upstream region of the PON1 locus correspond to the sequence coordinates of GenBank Accession No. AF051133. In addition, the genomic sequences encoding the PON1 gene and associated regions of non-coding DNA are provided in GenBank Accession No. AF539592.

### BRIEF DESCRIPTION OF THE FIGURES

FIG. 1 shows the nucleotide sequence of the upstream region of the PON1 locus. The nucleotide sequence coordinates are based on GenBank Accession No. AF051133. The position of the T/C polymorphism at nucleotide 911 is shown in bold. Positions of -107 Consensus primer (SEQ ID No. 5; nucleotides 654-671), PON1 SNP Primer 1 (SEQ ID No. 3; nucleotides 927-911) and PON1 SNP Primer 2 (SEQ ID No. 4; nucleotides 927-911) are also shown.

### GENERAL DESCRIPTION OF THE INVENTION

As discussed above, in one form the present invention provides a method of identifying a subject predisposed to lacunar stroke, the method including the step of identifying in the subject the presence of a thymine to cytosine mutation at position -107 in both alleles of the paraoxonase 1 locus.

In this study it has been found that there is an increased risk of lacunar stroke for individuals homozygous for the T to C polymorphism at -107 in the upstream region of PON1. In this regard, the structure of the human PON1 locus is as described in GenBank Accession No. AF539592. The coordinates for the position of the T/C polymorphism at position -107 are with respect to the ATG initiation codon.

It will be appreciated that this form of the present invention is also useful for identifying subjects in need of medical intervention to prevent and/or treat lacunar stroke.

Accordingly, in another form the present invention also provides a method of identifying a subject suitable for intervention to prevent and/or treat a lacunar stroke, the method including the step of identifying in the subject the presence of a thymine to cytosine mutation at position -107 in both alleles of the paraoxonase 1 locus.

Methods of medical intervention for the prevention and treatment of lacunar stroke are known in the art.

The subject is any human subject of either gender for which the predisposition to lacunar stroke is to be determined. Preferably, the subject is a human of Caucasian origin.

The identification of the -107 T to C mutation in a subject may be determined by a suitable method known in the art.

For example, DNA may be isolated from the subject by isolating genomic DNA from whole venous blood as described in Miller et al. (1988) Nucleic Acids Research 16(3):1215. To identify the mutation, DNA sequencing (either manual sequencing or automated fluorescent sequencing) can be used to detect the mutation. Another approach for identifying mutations is the single-stranded conformation polymorphism assay (SSCA) (as described in Orita et al. (1989) Genomics 5(4): 874-879. This method does not detect all sequence changes, especially if the DNA fragment size is greater than 200 bp, but can be optimized to detect most DNA sequence variation. Fragments which have shifted mobility on SSCA gels are then sequenced to determine the exact nature of the DNA sequence variation.

Another approach is based on the detection of mismatches between two complementary DNA strands, including clamped denaturing gel electrophoresis (as described in Sheffield et al. (1991) Am. J. Hum. Genet. 49:699-706), heteroduplex analysis (as described in White et al. (1992) Genomics 12:301-303) and chemical mismatch cleavage (as described in Grompe et al. (1989) Proc. Natl. Acad. Sci. USA 86:5855-5892). Once a specific mutation is identified, an allele specific detection approach such as allele specific oligonucleotide hybridization can be utilized.

If DNA sequence analysis is used to identify a mutation, the presence of a mutation in one allele (ie the subject is heterozygous for the mutation) will be by the presence of two nucleotides at the relevant position in the DNA sequence. Sequence of the DNA from a subject homozygous for the normal allele or homozygous for the mutation will yield only the presence of the appropriate nucleotide at the relevant position of the DNA sequence.

To provide a suitable template for sequencing, a region of the genomic DNA isolated from the subject may be amplified using appropriately designed primers. Sequencing reactions with an appropriate primer and the analysis of the DNA sequence may be performed by a suitable method known in the art. In the case of the mutation being the -107 T to C mutation in the upstream region of the PON1 locus, a preferred region for amplification is the region spanning nucleotides 654 to 927 of SEQ ID No. 1.

Alternatively, the presence of a mutation may be determined using sequence specific primers that will only amplify either the wild type allele or the allele with the mutation from the DNA isolated from the subject. If sequence specific primers are used to amplify the DNA, a consensus primer and one of two alternative primers will be used. Each of the alternative primers will have a 3′ terminal nucleotide that either corresponds to the wild type sequence (a WT primer) or the polymorphic sequence (a SNP primer). In this case, amplification will only occur from the template having the correct complementary nucleotide.

In this case, a preferred region of the PON1 locus for amplification with WT and SNP primers is the region spanning nucleotides 654 to 927 of SEQ ID No.1.

Accordingly, in another form the present invention provides an isolated nucleic acid, the polynucleotide consisting of the sequence spanning nucleotides 654 to 927 of SEQ ID No. 1.

The sequence of the region spanning nucleotides 654 to 927 of SEQ ID No.1 is designated SEQ ID No. 2 and is as follows:

5′-CAAGGAC CGGGATGGCA CAAAGTGAGT GCTCACCAAA
GCTTGACTGT CCTTTCCCAT GGCAATTTAC TTCAGCTTGT
TTGATTTCCC CTCCCCGACT GGACTAGGCA CCTATTCTCT
GTCTTCTCTC TTTACAGTTG GAAGGAGCAA AATGGGACTT
TTGGCTGAAA GTGCTGAGCT CCTGCGGTGG GGGCTGACCG
CAAGCCGCGC CTTCTGTGCA CCTGGTCGGC CCAGCTAGCT
GCGGACCCGG CGGGGAGGGG CCGGGCGGGC CAATCGG-3′

Preferably, the primers used to amplify the region of the PON1 locus containing the -107 C polymorphism are SEQ ID No. 3 (5′-CCGATTGGCCCGCCCCG-3′) and SEQ ID No. 5 (5′-CAAGGACCGGGATGGCAC-3′), and the primers used to amplify the region of the PON1 locus having the -107 T polymorphism are SEQ ID No. 4 (5′-CCGATTGGCCCGCCCCA-3′) and SEQ ID No. 5 (5′-CMGGACCGGGATGGCAC-3′). Suitable reaction conditions to amplify the PON1 locus with these primers include amplification using a PTC-200 Peltier Thermal Cycler (MJ Research) with the following PCR cycling parameters: 5 cycles of 96° C. for 25 seconds, 70° C. for 45 seconds, and 72° C. for 45 seconds; 21 cycles of 96° C. for 25 seconds, 65° C. for 50 seconds, and 72° C. for 45 seconds; 4 cycles of 96° C. for 25 seconds, 55° C. for 60 seconds, and 72° C for 125 seconds.

The amplification products may be detected by a suitable method known in the art. For example, the amplification products may be run on an agarose gel and stained with ethidium bromide for visualization.

Preferably, the identification of a mutation in the various forms of the present invention includes amplification of a region containing the mutation from a nucleic acid isolated or derived from the subject.

Other methods to identify mutations involve hybridization of nucleic acid containing the mutation with other nucleic acids (ie a reporter nucleic acid) that allows discrimination between differences in nucleic acid sequences. For example, Southern analysis with an oligonucleotide may be used to detect mutations. Alternatively, methods are known in the art in which the oligonucleotide is attached to a solid substrate, such as chip, and the binding of a nucleic acid containing a mutation detected by binding (or lack thereof) to the oligonucleotide. In these cases, the identification of a mutation in a subject also includes detection of the mutation by hybridisation of nucleic acid isolated or derived from the subject to a reporter nucleic acid.

Thus, the identification of a mutation in the various forms of the present invention may also include detection of the mutation by hybridization of nucleic acid isolated or derived from the subject to a reporter nucleic acid.

The present invention also provides a method of identifying a subject predisposed to small vessel occlusion, the method including the step of identifying in the subject the presence of a thymine to cytosine mutation at position -107 in both alleles of the paraoxonase 1 locus.

As will be appreciated, this form of the present invention is useful for identifying a subject in need of medical intervention to prevent and/or treat a small vessel occlusion.

Accordingly, this form of the present also provides a method of identifying a subject suitable for intervention to prevent and/or treat a small vessel occlusion, the method including the step of identifying in the subject the presence of a thymine to cytosine mutation at position -107 in both alleles of the paraoxonase 1 locus.

Methods of medical intervention for the prevention and treatment of small vessel occlusion are known in the art.

The subject is any human subject of either gender for which the predisposition to small vessel occlusion is to be determined. Preferably, the subject is a human of Caucasian origin.

The small vessel occlusion is any thrombotic or embolic occlusion that may occur in a small vessel in a subject, including a small vessel occlusion manifesting clinically as lacunar stroke, cerebral leukoaraiosis, dementia, cerebral leukoaraiosis, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the small vessel occlusion occurs in the brain. Most preferably, the small vessel occlusion is one manifesting clinically as a lacunar stroke.

The identification in the subject of the presence of a thymine to cytosine mutation at position -107 in both alleles of the PON1 locus may be by a suitable method known in the art, as described previously.

The present invention also provides a method of identifying a subject predisposed to developing a disease or condition associated with small vessel occlusion, the method including the step of identifying in the subject the presence of a thymine to cytosine mutation at position -107 in both alleles of the paraoxonase 1 locus.

As will be appreciated, this form of the present invention is useful for identifying a subject in need of medical intervention to prevent and/or treat the development of a disease or condition associated with small vessel occlusion.

Accordingly, in another form the present invention provides a method of identifying a subject suitable for intervention to prevent and/or treat a disease or condition associated with small vessel occlusion, the method including the step of identifying in the subject the presence of a thymine to cytosine mutation at position -107 in both alleles of the paraoxonase 1 locus.

Methods of medical intervention for the prevention and treatment of diseases and conditions associated with small vessel occlusion are known in the art.

The subject is any human subject of either gender for which the predisposition to developing a disease or condition associated with small vessel occlusion is to be determined. Preferably, the subject is a human of Caucasian origin.

Examples of diseases or conditions associated with small vessel occlusion include lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the disease or condition associated with small vessel occlusion is a disease or condition associated with small vessel occlusion in the brain. Most preferably, the disease or condition associated with small vessel occlusion is one manifesting clinically as a lacunar stroke.

The identification in the subject of the presence of a thymine to cytosine mutation at position -107 in both alleles of the PON1 locus may be by a suitable method known in the art, as described previously.

The present invention also provides a method of determining the risk of lacunar stroke in a subject, the method including the step of determining in the subject the presence of a thymine to cytosine mutation at position -107 in one or both alleles of the paraoxonase 1 locus.

The risk of lacunar stroke in a subject is the probability that a subject with a thymine to cytosine mutation at position -107 in one or both alleles of the PON1 locus may suffer a lacunar stroke, as compared to the probability that a subject in the general population may suffer a lacunar stroke, under circumstances where other risk factors (eg atrial fibrillation, history of smoking) for having an lacunar stroke between the subjects are the same.

In this regard, the presence of a thymine to cytosine mutation at position -107 in both alleles of the PON1 locus (ie an individual homozygous for the polymorphism) indicates an elevated risk that the subject may suffer a lacunar stroke, as compared to the risk of lacunar stroke for another subject in the general population, or as compared to the risk of a lacunar stroke in a subject having similar risk factors (eg atrial fibrillation, history of smoking).

A subject with a thymine to cytosine in one allele (ie a subject heterozygous for the polymorphism) will have a risk of suffering a lacunar stroke similar to that of a subject homozygous for the -107 T polymorphism.

The subject is any human subject of either gender for which the risk of suffering a lacunar stroke is to be determined. Preferably, the subject is a human of Caucasian origin.

Determination of the presence in the subject of a thymine to cytosine polymorphism at position -107 in one or both of the alleles of the PON1 locus may be by a suitable method known in the art, as described previously.

The present invention also provides a method of determining the risk of small vessel occlusion in a subject, the method including the step of determining in the subject the presence of a thymine to cytosine mutation at position -107 in one or both alleles of the paraoxonase 1 locus.

The risk of small vessel occlusion in a subject is the probability that a subject with a thymine to cytosine mutation at position -107 in one or both alleles of the PON1 locus may suffer a small vessel occlusion, as compared to the probability that a subject in the general population may suffer a small vessel occlusion, under circumstances where other risk factors (eg atrial fibrillation, history of smoking) for having a small vessel occlusion between the subjects are the same.

In this regard, the presence of a thymine to cytosine mutation at position -107 in both alleles of the PON1 locus (ie an individual homozygous for the polymorphism) indicates an elevated risk that the subject may suffer a small vessel occlusion as compared to a subject in the general population, or as compared to a subject having similar risk factors (eg atrial fibrillation, history of smoking).

A subject with a thymine to cytosine in one allele (ie a subject heterozygous for the polymorphism) will have a risk of suffering a small vessel occlusion similar to that of a subject homozygous for the -107 T polymorphism.

The subject is any human subject of either gender for which the risk of suffering a small vessel occlusion is to be determined. Preferably, the subject is a human of Caucasian origin.

The small vessel occlusion is any thrombotic or embolic occlusion that may occur in a small vessel in a subject, including small vessel occlusion manifesting clinically as lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the small vessel occlusion occurs in the brain. Most preferably, the small vessel occlusion is one manifesting clinically as a lacunar stroke.

Determination of the presence in the subject of a thymine to cytosine polymorphism at position -107 in one or both of the alleles of the PON1 locus may be by a suitable method known in the art, as described previously.

The present invention also provides a method of determining the risk of developing a disease or condition associated with small vessel occlusion in a subject, the method including the step of determining in the subject the presence of a thymine to cytosine mutation at position -107 in one or both alleles of the paraoxonase 1 locus.

The risk of developing a disease or condition associated with small vessel occlusion in a subject is the probability that a subject with a thymine to cytosine mutation at position -107 in one or both alleles of the PON1 locus may develop a disease or condition associated with small vessel occlusion, as compared to the probability that a subject in the general population may develop a disease or condition associated with small vessel occlusion, under circumstances where other risk factors (eg atrial fibrillation, history of smoking) between the subjects are the same.

In this regard, the presence of a thymine to cytosine mutation at position -107 in both alleles of the PON1 locus (ie an individual homozygous for the polymorphism) indicates an elevated risk that the subject may develop a disease or condition associated with small vessel occlusion as compared to the probability that a subject in the general population, or or as compared to a subject having similar risk factors (eg atrial fibrillation, history of smoking).

A subject with a thymine to cytosine in one allele (ie a subject heterozygous for the polymorphism) will have a risk of developing a disease or condition associated with small vessel occlusion similat to that of a subject homozygous for the -107 T polymorphism.

The subject is any human subject of either gender for which the risk of suffering a lacunar stroke is to be determined. Preferably, the subject is a human of Caucasian origin.

Examples of diseases or conditions associated with small vessel occlusion include lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the disease or condition associated with small vessel occlusion is a disease or condition associated with small vessel occlusion in the brain. Most preferably, the disease or condition associated with small vessel occlusion is a lacunar stroke.

Determination of the presence in the subject of a thymine to cytosine polymorphism at position -107 in one or both of the alleles of the PON1 locus may be by a suitable method known in the art, as described previously.

The present invention also provides a method of identifying a subject suitable for treatment with an agent that decreases the activity of paraoxonase 1, the method including the step of determining in the subject the presence of a thymine to cytosine mutation at position -107 in both alleles of the paraoxonase 1 locus.

The subject is any human subject of either gender for whom the treatment with an agent that decreases the activity of paraoxonase 1 may be beneficial. Preferably, the subject is a human of Caucasian origin.

In this regard, subjects that may benefit from the treatment with an agent that decreases the activity of paraoxonase 1 are subjects at increased risk of lacunar stroke, at increased risk of small vessel occlusion including a small vessel occlusion occurring in the brain, or at increased risk of developing a disease or condition associated with small vessel occlusion including a disease or condition associated with small vessel occlsuion in the brain, and small vessel occlusion manifesting clinically dementia, cerebral leukoaraiosis, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

In this form of the present invention, the presence of a thymine to cytosine mutation at position -107 in both alleles of the PON1 locus (ie an individual homozygous for the polymorphism) may be used to identify a subject suitable for treatment with an agent that decreases the activity of paraoxonase 1.

A subject with a thymine to cytosine in one allele (ie a subject heterozygous for the polymorphism), or a subject homozygous for the thymine allele, are subjects that are unlikey to be benefit from treatment with an agent that decreases the activity of paraoxonase 1.

The identification in the subject of the presence of a thymine to cytosine mutation at position -107 in one or both alleles of the PON1 locus may be by a suitable method known in the art, as described previously.

The present invention also provides a method of treating a subject susceptible to suffering a lacunar stroke, the method including the step of administering to the subject an effective amount of an agent that decreases the activity of paraoxonase 1.

The subject is any human subject of either gender susceptible to a lacunar stroke. Preferably, the subject is a human of Caucasian origin.

The agent is any agent that when administered to a subject has the capacity to decrease the activity of paraoxonase 1.

The effective amount of the agent that decreases the activity of paraoxonase 1 to be administered to a subject is not particularly limited, so long as it is within such an amount and in such a form that generally exhibits a pharmacologically useful or therapeutic effect.

In this regard, an effective amount of the agent that decreases the activity of PON1 may be appropriately chosen, depending upon for example the extent of decrease in the activity of PON1 to be achieved in the subject, the age and body weight of the subject, the frequency of administration, and the presence of other active agents.

The administration of the agent that decreases the activity of PON1 may be within any time suitable to produce the desired effect of decreasing the activity of PON1 in the subject, and may be administered orally, parenterally or by any other suitable means, and therefore transit time of the drug must be taken into account.

The administration of the agent that decreases the activity of PON1 may also include the use of one or more pharmaceutically acceptable additives, including pharmaceutically acceptable salts, amino acids, polypeptides, polymers, solvents, buffers, excipients and bulking agents, taking into consideration the particular physical and chemical characteristics of the agent that decreases the activity of PON1.

For example, the agent can be prepared into a variety of pharmaceutical preparations in the form of, e.g., an aqueous solution, an oily preparation, a fatty emulsion, an emulsion, a gel, etc., and these preparations can be administered as intramuscular or subcutaneous injection or as injection to the organ, or as an embedded preparation or as a transmucosal preparation through nasal cavity, rectum, uterus, vagina, lung, etc. The composition may be administered in the form of oral preparations (for example solid preparations such as tablets, capsules, granules or powders; liquid preparations such as syrup, emulsions or suspensions). Compositions containing the agent may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The administration of the agent may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the agent.

For these purposes, the composition may be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile injectable form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

When administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding the active ingredient optionally with one or more accessory ingredients. Compressed tablets may be prepared by compressing, in a suitable machine, the active ingredient in a free- flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

The administration of the agent may also utilize controlled release technology. The agent may also be administered as a sustained-release pharmaceutical. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the agent may be incorporated into a hydrophobic polymer matrix for controlled release over a period of days. The composition of the invention may then be molded into a solid implant, or externally applied patch, suitable for providing efficacious concentrations of the agent over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The composition may then be molded into a solid implant suitable for providing efficacious concentrations of the agent over a prolonged period of time without the need for frequent re-dosing. The agent can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant.

The present invention also provides a method of treating a subject susceptible to small vessel occlusion or a disease or condition associated with small vessel occlusion, the method including the step of administering to the subject an effective amount of an agent that decreases the activity of paraoxonase 1.

The subject is any human subject of either gender susceptible to small vessel occlusion or a disease or condition associated with small vessel occlusion. Preferably, the subject is a human of Caucasian origin.

Preferably, the small vessel occlusion is a small vessel occlusion occurring in the brain, such as a small vessel occlusion manifesting clinically as a lacunar stroke.

Example of diseases or conditions associated with small vessel occlusion include lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the disease or condition associated with small vessel occlusion is a disease or condition associated with a small vessel occlusion in the brain. Most preferably, the disease or condition is a lacunar stroke.

The agent is any agent that when administered to a subject has the capacity to decrease the activity of paraoxonase 1. Methods for administration are as previously described.

The effective amount of the agent that decreases the activity of paraoxonase 1 to be administered to a subject is not particularly limited, so long as it is within such an amount and in such a form that generally exhibits a pharmacologically useful or therapeutic effect. Methods of administering the agent have been discussed previously.

In this regard, an effective amount of the agent that decreases the activity of PON1 may be appropriately chosen, depending upon for example the extent of decrease in the activity of PON1 to be achieved in the subject, the types of diseases or conditions associated with small vessel occlusion to be treated, the age and body weight of the subject, the frequency of administration, and the presence of other active agents.

The administration of the agent that decreases the activity of PON1 may be within any time suitable to produce the desired effect of decreasing the activity of PON1 in the subject, and may be administered orally, parenterally or by any other suitable means, and therefore transit time of the drug must be taken into account.

The present invention also provides a method of identifying a subject predisposed to lacunar stroke, the method including the step of identifying an arylesterase activity of paraoxonase 1 in serum from the subject of greater than 100 units per ml.

In this form of the present invention, an arylesterase activity of PON1 of greater than 100 units per ml in serum will indicate that the subject has a greater risk of suffering a lacunar stroke, as compared to a subject in the general population, under circumstances where other risk factors (eg atrial fibrillation, history of smoking) for having a lacunar stroke between the subjects are the same.

The subject is any human subject of either gender for which the predisposition to lacunar stroke is to be determined. Preferably, the subject is a human of Caucasian origin.

The identification of an arylesterase activity of paraoxonase 1 in serum from the subject in the various forms of the present invention may be determined by a suitable method known in the art.

For example, the arylesterase activity may determined essentially as described in Furlong et al. (1993) Chem Biol Interact 87:35-48 or Blattner Garin et al. (1994). Biochem J. 304:549-554.

As will be appreciated, in determining the level of arylestease activity of PON1 in serum in the various forms of the present invention, sufficient repetitions of the measurement are required to establish a statistically significant mean value. In addition, to ensure reproducibility between assays, an internal standard (eg serum of known activity) should be used.

The present invention also provides a method of identifying a subject predisposed to small vessel occlusion, the method including the step of identifying an arylesterase activity of paraoxonase 1 in serum from the subject of greater than 100 units per ml.

In this form of the present invention, an arylesterase activity of PON1 of greater than 100 units per ml in serum from a subject will indicate that the subject has a greater risk of suffering a small vessel occlusion, as compared to a subject in the general population, under circumstances where other risk factors (eg atrial fibrillation, history of smoking) for having a small vessel occlusion between the subjects are the same.

The small vessel occlusion is any thrombotic or embolic occlusion that may occur in a small vessel in a subject, including small vessel occlusion manifesting clinically as lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the small vessel occlusion occurs in the brain. Most preferably, the small vessel occlusion manifests clinically as a lacunar stroke.

The subject is any human subject of either gender for which the predisposition to small vessel occlusion is to be determined. Preferably, the subject is a human of Caucasian origin.

The present invention also provides a method of identifying a subject predisposed to developing a disease or condition associated with small vessel occlusion, the method including the step of identifying an arylesterase activity of paraoxonase 1 in serum from the subject of greater than 100 units per ml.

In this form of the present invention, an arylesterase activity of PON1 of greater than 100 units per ml in serum from a subject will indicate that the subject has a greater risk of developing a disease or condition associated with small vessel occlusion, as compared to a subject in the general population, under circumstances where other risk factors (eg atrial fibrillation, history of smoking) for developing a disease or condition associated with small vessel occlusion between the subjects are the same.

Examples of diseases or conditions associated with small vessel occlusion include lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the disease or condition associated with small vessel occlusion occurs in the brain. Most preferably, the disease or condition associated with small vessel occlusion is a lacunar stroke.

The subject is any human subject of either gender for which the predisposition to small vessel occlusion is to be determined. Preferably, the subject is a human of Caucasian origin.

The present invention also provides a method of determining the risk of lacunar stroke in a subject, the method including the step of identifying an arylesterase activity of paraoxonase 1 in serum from the subject of greater than 100 units per ml.

The risk of lacunar stroke in a subject is the probability that a subject with an erylesterase activity of paraoxonase 1 in serum of greater than 100 units per ml may suffer a lacunar stroke, as compared to the probability that a subject in the general population may suffer a lacunar stroke, under circumstances where other risk factors (eg atrial fibrillation, history of smoking) for having an lacunar stroke between the subjects are the same.

The subject is any human subject of either gender for which the risk of suffering a lacunar stroke is to be determined. Preferably, the subject is a human of Caucasian origin.

The present invention also provides a method of determining the risk of small vessel occlusion in a subject, the method including the step of identifying an arylesterase activity of paraoxonase 1 in serum from the subject of greater than 100 units per ml.

The risk of small vessel occlusion in a subject is the probability that a subject with an erylesterase activity of paraoxonase 1 in serum of greater than 100 units per ml may suffer a small vessel occlusion, as compared to the probability that a subject in the general population may suffer a small vessel occlusion, under circumstances where other risk factors (eg atrial fibrillation, history of smoking) for having a small vessel occlusion between the subjects are the same.

The subject is any human subject of either gender for which the risk of suffering a small vessel occlusion is to be determined. Preferably, the subject is a human of Caucasian origin.

The small vessel occlusion is any thrombotic or embolic occlusion that may occur in a small vessel in a subject, including small vessel occlusion manifesting clinically as lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the small vessel occlusion occurs in the brain. Most preferably, the small vessel occlusion is one manifesting clinically as a lacunar stroke.

The present invention also provides a method of determining the risk of a subject developing a disease or condition associated with small vessel occlusion, the method including the step of identifying an arylesterase activity of paraoxonase 1 in serum from the subject of greater than 100 units per ml.

The risk of developing a disease or condition associated with small vessel in a subject is the probability that a subject with an arylesterase activity of paraoxonase 1 in serum of greater than 100 units per ml may develop a disease or condition associated with small vessel occlusion, as compared to the probability that a subject in the general population may develop a disease or condition associated with small vessel occlusion, under circumstances where other risk factors (eg atrial fibrillation, history of smoking) for developing a disease or condition associated with small vessel occlusion between the subjects are the same.

The subject is any human subject of either gender for which the risk of developing a disease or condition associated with small vessel occlusion is to be determined. Preferably, the subject is a human of Caucasian origin.

The disease or condition associated with small vessel occlusion is disease or condition arising from a thrombotic or embolic occlusion in a small vessel in a subject, including lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

Preferably, the diease or consition associated with small vessel occlusion is a disease or condition associated with small vessel occlusion in the brain. Most preferably, the disease or condition associated with small vessel occlusion is a lacunar stroke.

The present invention also provides a method of identifying a subject suitable for treatment with an agent that decreases the activity of paraoxonase 1, the method including the step of identifying an arylesterase activity of paraoxonase 1 in serum from the subject of greater than 100 units per ml.

In this form of the present invention, an arylseterase activity of paraoxonase 1 in excess of 100 units per ml in serum identifies a subject as being suitable for treatment with an agent that decreases the activity of paraoxonase 1.

The subject is any human subject of either gender for whom the treatment with an agent that decreases the activity of paraoxonase 1 may be beneficial. Preferably, the subject is a human of Caucasian origin.

In this regard, subjects that may benefit from the treatment with an agent that decreases the activity of paraoxonase 1 are subjects at increased risk of lacunar stroke, at increased risk of small vessel occlusion or developing a disease or condition associated with small vessel occlusion, including small vessel occlusion manifesting clinically as lacunar stroke, cerebral leukoaraiosis, dementia, ischemic heart disease (including ischemic cardiomyopathy), peripheral vascular disease, disseminated intravascular coagulation, small vessel vasculitis, ischemic neuropathy, ischemic retinopathy, ischemic gastropathy (including small and large bowel ischemia), diffuse pulmonary embolism and vascular impotence.

The present invention also provides a method of identifying an agent for treating a subject susceptible to lacunar stroke, small vessel occlusion or a disease or condition associated with small vessel occlusion, the method including the steps of:
(a) exposing an agent to a cell expressing PON1, wherein the cell includes a mutation that results in overexpression of PON1 compared to a cell without the mutation;
(b) determining the level of expression and/or activity of PON1 from the cell; and
(c) identifying the agent as an agent capable of decreasing the expression and/or activity of PON1.

This form of the present invention is directed to the identification of agents by this method that are capable of decreasing the expression and/or activity of PON1. Agents so identified are candidate compounds for administering to a subject susceptible to lacunar stroke, small vessel occlusion or a disease or condition associated with small vessel occlusion.

The term “exposing” is be understood to include within its scope the external administration of the agent to a cell or the intracellular expression of the agent in the cell. Accordingly, the exposing of an agent to a cell may be by way of contacting the cell with the agent, or for example, by way of transforming the cell with a recombinant nucleic acid capable of directing the expression of the agent in the cell.

The cell is any cell having a mutation that results in the overexpression of PON1, as compared to a similar cell not having the mutation.

Preferably the cell is a liver cell. Most preferably, the liver cell is a human liver cell. An example of a suitable liver cell is a Hep G2 cell.

The mutation in the cell is any mutation that results in an overexpression of PON1. For example, the mutation may be in a gene or region not associated with the PON1 locus, or be a mutation in the PON1 locus, such as a mutation in a regulatory region upstream of the PON1 coding region, a mutation in an exon of the PON1 gene, a mutation in an intron that affects splicing, or a mutation in the 3′ region of the PON1 that affects translation or mRNA stability.

The mutation may be present in one or both alleles of the particular gene. Preferably, the mutation is present in both alleles of the particular gene.

The ability of a mutation to increase the expression of PON1 may be confirmed by a suitable method known in the art. For example, the ability of specific mutation to increase overexpression may be determined by assaying for PON1 levels using a PON1 specific antibody, or by measuring the enzymatic activity of PON1.

Preferably, the mutation in the cell is a mutation in an upstream region of the PON1 gene. More preferably, the mutation is a mutation in an enhancer element in the PON1 gene. Most preferably, the mutation is a thymine to cytosine mutation in both alleles at position -107 of the upstream region of the PON1 locus.

As will be appreciated, the ability of an agent to decrease the expression and/or activity of PON1 will depend on the concentration of the agent exposed to the cell. Accordingly, the agent will be exposed to the cell at a suitable concentration for testing the ability of the agent at that concentration to decrease the expression and/or activity of PON1.

Agents that decrease the expression and/or activity of PON1 may be identified by comparison expression levels and/or activity in the absence of the agent. An agent capable of decreasing the expression and/or activity of PON1 so identified is a candidate compound for administering to subject to reduce the likelihood of the subject suffering a lacunar stroke, small vessel occlusion or a disease or condition associated with small vessel occlusion.

The present invention also provides a method of identifying an agent for treating a subject susceptible to a lacunar stroke, small vessel occlusion or a disease or condition associated with small vessel occlusion, the method including the steps of:
(a) exposing an agent to a cell transformed with all or part of the PON1 locus, wherein the transformed locus includes a thymine to cytosine mutation at position -107 and the transformed locus regulates expression of a reporter gene;
(b) determining the level of expression of the reporter gene in the cell so exposed to the agent; and
(c) identifying the agent as an agent that decreases PON1 expression by the decrease in expression of the reporter gene.

This form of the present invention is directed to the identification of agents by this method that are capable of decreasing expression of PON1. Agents so identified are candidate compounds for administering to a subject to reduce the likelihood of the subject suffering a lacunar stroke, a small vessel occlusion or developing a disease or condition associated with small vessel occlusion.

The cell is any cell transformed with all or part of the PON1 locus having a thymine to cytosine mutation at position -107 and regulating the expression of a suitable reporter gene.

Examples of a suitable cell type that may be transformed include a liver cell, for example a Hep G2 cell.

The DNA coding for all or part of the PON1 locus which is to be used to drive the expression of a reporter gene may be derived from an appropriate human genomic clone, the clone produced by a suitable method known in the art.

For example, the PON1 locus having the T to C mutation at position -107 may be cloned from genomic DNA by performing a partial digestion of the DNA with a restriction enzyme (eg Sau 3A1) and shot gun cloning into a lambda or cosmid vector as described in Sambrook, J, Fritsch, E. F. and Maniatis, T. Molecular Cloning: A Laboratory Manual 2nd. ed. Cold Spring Harbor Laboratory Press, New York. (1989). The library may be screened with an appropriate probe (eg a labelled oligonucleotide or a nick translated fragment from the upstream region of the PON1 locus) to isolated an appropriate clone, as also described in Sambrook et al. (1998).

Alternatively, all or part of the PON1 locus may be generated by PCR amplification from the genomic DNA, using appropriate primers, as described in as described in Dieffenbach, C. W. and G. S. Dveksler (1995) PCR Primer, a Laboratory Manual, Cold Spring Harbor Press, Plainview, N.Y.

The DNA containing the region for cloning may then be fused to an appropriate reporter gene by standard cloning protocols, as described in Sambrook, J, Fritsch, E. F. and Maniatis, T. Molecular Cloning: A Laboratory Manual 2nd. ed. Cold Spring Harbor Laboratory Press, New York. (1989)

Examples of suitable reporter genes include the chloramphenicol acetyltransferase (CAT) gene, green fluorescent protein (and variants thereof), and luciferase.

Transfection of cells with the reporter gene constructs may be performed by a suitable method known in the art, including the calcium phosphate precipitation method, incorporating a glycerol shock, as described in Sambrook, J, Fritsch, E. F. and Maniatis, T. Molecular Cloning: A Laboratory Manual 2nd. ed. Cold Spring Harbor Laboratory Press, New York. (1989). After exposure, cells may be harvested and relative changes in reporter gene activity quantitated.

Transfection efficiency may also be evaluated by cotransfecting the cells with an appropriate control construct, such as the cytomegalovirus promoter and lac Z gene construct. β-Galactosidase assay may then be performed as described in Sambrook, J, Fritsch, E. F. and Maniatis, T. Molecular Cloning: A Laboratory Manual 2nd. ed. Cold Spring Harbor Laboratory Press, New York. (1989), and may be taken as a direct index of the efficiency of transfection and used to normalize reporter gene activities among various experiments.

An agent that is capable of decreasing the expression of the reporter gene may then be tested for its ability to decrease the expression of PON1. As will be appreciated, the ability of an agent to increase the expression of the reporter gene will depend on the concentration of the agent exposed to the cell. Accordingly, the agent will be exposed to the cell at a suitable concentration for testing the ability of the agent at that concentration to decrease the expression of the reporter gene.

The ability of the agent to decrease the expression of PON1 may then be determined by a suitable method known in the art.

The present invention also provides an isolated nucleic acid consisting of the sequence according to SEQ. ID No.3 or RNA equivalent thereof.

SEQ ID NO:3 has the sequence 5′-CCGATTGGCCCGCCCCG-3′, and corresponds to the nucleotide sequence of the PON1 SNP Primer 1.

The nucleic acid in the various forms of the present invention may be synthesized and purified by a standard method known in the art. For example, phosphorothioate oligonucleotides may be synthesized by the method as described in Stein et al. (1988) Nucl. Acids Res. 16: 3209.

The present invention also provides an isolated nucleic acid with one or more base substitutions in the sequence according to SEQ ID No. 3, wherein the nucleic acid hybridises with the complement of SEQ ID No.3 under stringent hybridisation conditions and the stringent hybridisation conditions include hybridisation in 6×SSC at 42° C. and washing in 2×SSC at 20° C.

This form of the present invention contemplates an isolated nucleic acid with one or more base substitutions of SEQ ID NO:3 which hybridises with the complement of SEQ ID No.3 under stringent hybridisation conditions, wherein the stringent reaction conditions include hybridisation in 6×SSC at 42° C. and washing in 2×SSC at 20° C.

Stringent hybridisation conditions are conditions that allow complementary nucleic acids to bind to each other within a range from at or near the Tm (Tm is the melting temperature) to about 20° C. below Tm.

Factors such as the length of the complementary regions, type and composition of the nucleic acids (DNA, RNA, base composition), and the concentration of the salts and other components (e.g. the presence or absence of formamide, dextran sulfate and/or polyethylene glycol) must all be considered, essentially as described in in Current Protocols in Molecular Biology, John Wiley & Sons, N.Y. (1989).

For example, conditions that allow the nucleic acid to hybridise with the complement of SEQ ID No.3 under stringent conditions are as follows: prehybridization may be performed in a prehybridization solution (eg 6×SSC (1×=150 mM NaCl, 15 mM sodium citrate, pH 7.0), 5× Denhardt's reagent (1 g/l each of Ficoll, Polyvinyl-pyrrolidone, Bovine Serum Albumin), 1.0% SDS, 100 ug/mI denatured, fragmented salmon sperm DNA) for 2 to 12 hours. Hybridizition of the probe with the target (eg filter) may then be performed under conditions such as 6×SSC, 1.0% SDS, 100 ug/ml denatured, fragmented salmon sperm DNA, at 42° C. overnight, followed by washing with 2×SSC and 0.5% SDS at room temperature for 15 min at 20° C.

The present invention also provides an isolated nucleic acid consisting of the sequence according to SEQ. ID No.4 or RNA equivalent thereof.

SEQ ID NO:4 has the sequence 5′-CCGATTGGCCCGCCCCA-3′, and corresponds to the nucleotide sequence of the PON1 SNP Primer 2.

The present invention also provides an isolated nucleic acid with one or more base substitutions in the sequence according to SEQ ID No. 4, wherein the nucleic acid hybridises with the complement of SEQ ID No. 4 under stringent hybridisation conditions and the stringent hybridisation conditions include hybridisation in 6×SSC at 42° C. and washing in 2×SSC at 20° C.

For example, conditions that allow the nucleic acid to hybridise with the complement of SEQ ID No.4 under stringent conditions are as follows: prehybridization may be performed in a prehybridization solution (eg 6×SSC (1×=150 mM NaCl, 15 mM sodium citrate, pH 7.0), 5× Denhardt's reagent (1 g/l each of Ficoll, Polyvinyl-pyrrolidone, Bovine Serum Albumin), 1.0% SDS, 100 ug/ml denatured, fragmented salmon sperm DNA) for 2 to 12 hours.

Hybridizition of the probe with the target (eg filter) may then be performed under conditions such as 6×SSC, 1.0% SDS, 100 ug/ml denatured, fragmented salmon sperm DNA, at 42° C. overnight, followed by washing with 2×SSC and 0.5% SDS at room temperature for 15 min at 20° C.

The present invention also provides an isolated nucleic acid consisting of the sequence according to SEQ. ID No.5 or RNA equivalent thereof.

SEQ ID NO:5 has the sequence 5′-CMGGACCGGGATGGCAC-3′, and corresponds to the nucleotide sequence of the -107 Consensus primer.

The present invention also provides an isolated nucleic acid with one or more base substitutions in the sequence according to SEQ ID No. 5, wherein the nucleic acid hybridises with the complement of SEQ ID No. 5 under stringent hybridisation conditions and the stringent hybridisation conditions include hybridisation in 6×SSC at 42° C. and washing in 2×SSC at 20° C.

For example, conditions that allow the nucleic acid to hybridise with the complement of SEQ ID No.5 under stringent conditions are as follows: prehybridization may be performed in a prehybridization solution (eg 6×SSC (1×=150 mM NaCl, 15 mM sodium citrate, pH 7.0), 5× Denhardt's reagent (1 g/l each of Ficoll, Polyvinyl-pyrrolidone, Bovine Serum Albumin), 1.0% SDS, 100 ug/ml denatured, fragmented salmon sperm DNA) for 2 to 12 hours. Hybridizition of the probe with the target (eg filter) may then be performed under conditions such as 6×SSC, 1.0% SDS, 100 ug/mI denatured, fragmented salmon sperm DNA, at 42° C. overnight, followed by washing with 2×SSC and 0.5% SDS at room temperature for 15 min at 20° C.

The present invention also provides an isolated nucleic acid with one or more base substitutions in the sequence according to SEQ ID No. 3, wherein the nucleic acid has at least 80% homology to SEQ. ID No.3 or RNA equivalent thereof.

This form of the present invention contemplates an isolated nucleic acid with one or more substitutions in the sequence of SEQ ID No.3, the nucleic acid having at least 80% homology to SEQ ID No.3 or RNA equivalent thereof. Various algorithms exist for determining the degree of homology between any two nucleic acid sequences. For example, the BLAST algorithm can be used for determining the extent of sequence homology between two sequences. BLAST identifies local alignments between two sequences and predicts the probability of the local alignment occurring by chance. The BLAST algorithm is as described in Altschul et al., 1990, J. Mol. Biol. 215:403-410.

Preferably, the nucleic has at least 90% homology to SEQ. ID No.3 or RNA equivalent thereof. Most preferably, the nucleic has at least 95% homology to SEQ. ID No.3 or RNA equivalent thereof.

The present invention also provides an isolated nucleic acid with one or more base substitutions in the sequence according to SEQ ID No.4, wherein the nucleic acid has at least 80% homology to SEQ. ID No.4 or RNA equivalent thereof.

This form of the present invention contemplates an isolated nucleic acid with one or more substitutions in the sequence of SEQ ID No.4, the nucleic acid having at least 80% homology to SEQ ID No.4 or RNA equivalent thereof.

Once again, various algorithms exist for determining the degree of homology between any two nucleic acid sequences, as described previously.

Preferably, the nucleic has at least 90% homology to SEQ. ID No.4 or RNA equivalent thereof. Most preferably, the nucleic has at least 95% homology to SEQ. ID No.4 or RNA equivalent thereof.

The present invention also provides an isolated nucleic acid with one or more base substitutions in the sequence according to SEQ ID No.5, wherein the nucleic acid has at least 80% homology to SEQ. ID No.5 or RNA equivalent thereof.

This form of the present invention contemplates an isolated nucleic acid with one or more substitutions in the sequence of SEQ ID No. 5, the nucleic acid having at least 80% homology to SEQ ID No. 5 or RNA equivalent thereof.

Once again, various algorithms exist for determining the degree of homology between any two nucleic acid sequences, as described previously.

Preferably, the nucleic has at least 90% homology to SEQ. ID No.5 or RNA equivalent thereof. Most preferably, the nucleic has at least 95% homology to SEQ. ID No.5 or RNA equivalent thereof.

### Description of the Preferred Embodiments

Reference will now be made to experiments that embody the above general principles of the present invention. However, it is to be understood that the following description is not to limit the generality of the above description.

### EXAMPLE 1

Isolation of Genomic DNA

Genomic DNA was isolated from six millilitres of whole venous blood as described in Miller, S. A., D. D. Dykes, and H .F. Polesky (1988). “A simple salting out procedure for. extracting DNA from human nucleated cells” Nucleic Acids Research 16(3): 1215.

### EXAMPLE 2

Oligonucleotide Primers

The sequence of the two PON1-107 reverse allele-specific primers and the PON1-107 consensus primer is as follows:

| | |
|---|---|
| PON1 SNP Primer 1: | |
| 5′- CCGATTGGCCCGCCCCG -3′; | (SEQ ID No. 3) |
| PON1 SNP Primer 2: | |
| 5′- CCGATTGGCCCGCCCCA -3′, | (SEQ ID No. 4) |
| −107 Consensus primer: | |
| 5′- CAAGGACCGGGATGGCAC -3′. | (SEQ ID NO. 5) |

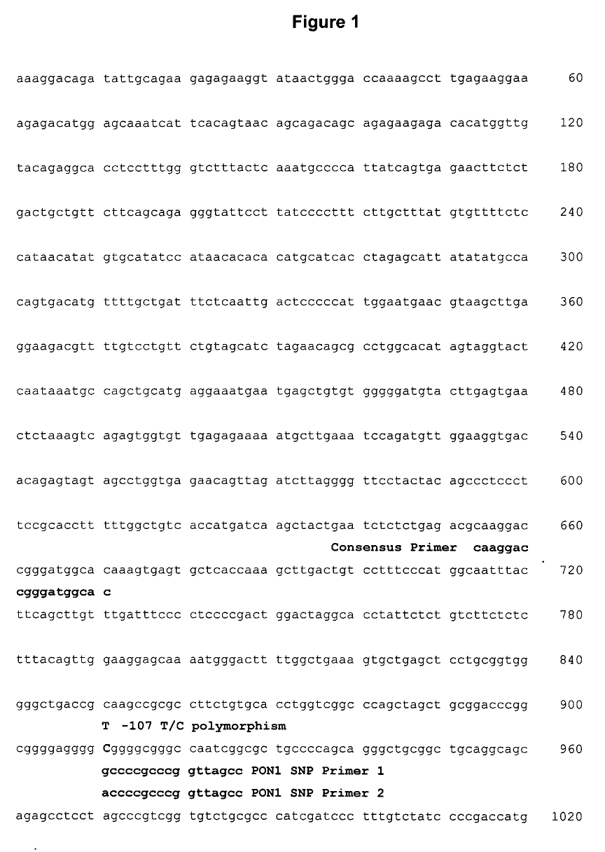

The relative position of the primers in the DNA sequence is shown in FIG. 1. These primers result in a 274 base pair DNA amplified fragment (spanning nucleotides 654-927 of GenBank Accession No. AF051133; SEQ ID No. 1) of the PON1 upstream region. The ATG inititiation codon is at position 1018 and transcription is likely to initiate at position 968.

The sequence of the two positive control primers coding for a 600 bp fragment of the HLA-DRB3 gene is as follows:

| | |
|---|---|
| Forward (sense) primer: | 5′-TGCCAAGTGGAGCACCCAA-3′ |
| Reverse (anti-sense) | 5′-GCATCTTGCTCTGTGCAGAT-3′ |
| primer: | |

The PON1 gene also has a polymorphism associated with amino acid 54 (referred to as M54L), resulting in either a methionine or leucine amino acid at this position. The sequence of the two reverse allele-specific primers for the PON1 M54L polymorphism and the consensus primer is as follows:

| | |
|---|---|
| PON1 M54L Primer 1: | 5′- CAGAAACTGGCTCTGAAGACA -3′; |
| PON1 M54L Primer 2: | 5′- CAGAAACTGGCTCTGAAGACT -3′; |
| M54L Consensus | 5′- AAGTGGGCATGGGTATACAG -3′. |
| primer: | |

PON1 M54L Primer 1 and 2 are located at position 48 to 68 of the DNA sequence of GenBank Accession No. U55879. The M54L Consensus Primer is located at postion 306-287 of GenBank Accession No. S56546.

All oligonucleotide primers were manufactured by Geneworks Pty Ltd. Primers were reconstituted with deionised and autoclaved water to a final concentration of 2000 μg/ml.

### EXAMPLE 3

Genotype Determination

A 274 base pair DNA fragment (spanning nucleotides 654-927 of GenBank Accession No. AF051133; SEQ ID No.1) of the PON1 upstream regulatory was amplified using the sequence-specific primer polymerase chain reaction method (PCR-SSP) as described in Bunce, M., C. M. O'Neill, M.C. Barnardo, et al. (1995) “Phototyping: comprehensive DNA typing for HLA-A, B, C, DRB1, DRB3, DRB4, DRB5 & DQB1 by PCR with 144 primer mixes utilizing sequence-specific primers (PCR-SSP)” Tissue Antigens 46(5): p. 355-367.

Similarly, A 150 base pair DNA fragment of the PON1 coding region was amplified using PON1 M54L Primer 1 and 2 and the M54L Consensus Primer with the same sequence-specific primer polymerase chain reaction method (PCR-SSP).

The specificity of PCR-SSP is derived from matching the terminal 3′-nucleotide of a primer with the target DNA sequence. Successful amplification by Taq polymerase during the PCR cycle will therefore only occur when matching between primer and DNA template (including the polymorphic nucleotide) occurs. Using this principle, oligonucleotide primers can be specifically tailored to incorporate a matching terminal 3′-nucleotide for a polymorphic site. A consensus primer that is complimentary to a stable, non-polymorphic region approximately 300-400 base pairs up or down stream in the DNA sequence allows for the amplification of a segment containing the single nucleotide polymorphism (snp). Under pre-specified PCR conditions, the amplification of the target DNA segment only occurs when both allele-specific and consensus primers are complimentary to the DNA template. The presence or absence of the snp can therefore be determined by detecting the PCR products using gel electrophoresis and visualisation by ethidium bromide incorporation. PCR-SSP works because the Taq polymerase is deficient in exonuclease activity and therefore is unable to repair a mismatched 3′-terminal primer nucleotide.

This methodology allows PCR multiplexing to be readily performed. Multiplexing involves the addition of two other primers in the same reaction mix that are complimentary to a different DNA site. In this study, two oligonucleotide primers giving rise to a 600 bp DNA fragment from the HLA-DRB3 gene were amplified in each PCR-SSP reaction as a positive control to discriminate between a failed and a negative PCR reaction. Without such a control, all homozygous results (eg negative reaction in wild-type allele) would be questionable. Another important feature of this method is that the primer design allows for multiple small volume PCR reactions (where each reaction is specific for an allele) to occur under the same PCR cycling conditions. Genotyping in this study was performed using a 96-well PCR plate.

(i) Reagents for SSP-PCR

| Reagent | Supplier |
|---|---|
| Ammonium Sulphate | BDH Laboratory Supplies |
| AmpliTaq DNA Polymerase, 5 U/μl | Applied Biosystems |
| Cresol Red Solution (10 mg/ml) | Fluka |
| dnTP's (nucleotides) | Applied Biosystems |
| Magnesium Chloride (25 mM) | Applied Biosystems |
| Trizma Base | Sigma Chemicals |
| Tween 20 | BDH Laboratory Supplies |

(ii) Oligonucleotide Primer Preparation

A primer stock containing the HLA-DRB3 positive control primers was made using the following protocol:

8 ml deionised and autoclaved water

50 μl cresol red

20 μl HLA-DRB3 forward primer

20 μl HLA-DRB3 reverse primer

Cresol red is an acid-base colour indicator (turns pink) when exposed to acidic DNA and allows for the visual identification of DNA contamination. The control primer stock is dispensed in separate 1.5 ml eppendorf tubes together with an allele-specific and consensus primer to a final volume of 1 ml.

5 μl of each primer mix was then dispensed in separate wells of a 96-well PCR plate. 10 μl of paraffin oil was added to each well to minimise primer and reagent evaporation during PCR thermal cycling.

The primer reaction mix for the PON1-107 T/C amd PON1 M54L SNPs was produced as follows by mixing 10 μl of allele-specific primer, 10 μl of the consensus primer and 980 ul of the control primer stock.

(iii) Reagent Mixes

10× PCR Buffer

Disolve 40.568 g Tris Base in 400 ml autoclaved water

Adjust pH to 8.9 with concentrated hydrochloric acid

Dissolve 10.96 g ammonium sulphate in the above solution

Autoclave

Add 5 ml Tween 20 and autoclaved water to 500 ml

10×dNTP

60 μl 100 mM dATP

60 μl 100 mM dCTP

αμl 100 mM dTTP

60 μl 100 mM dGTP

5760 μl autoclaved, deionised water.

TMDH Mixture

6 ml 10× PCR buffer

6 ml 10×dNTP

5.1 ml 25 mM Magnesium Chloride

6 ml autoclaved, deionised water

The PCR solution was prepared using the following protocol:

100 μl TMDH

57 μl autoclaved, deionised water

0.85 μl AmpliTaq DNA Polymerase (5U/μl)

3 μl DNA 1 μg/μl

8 μl of DNA solution was then added to each well containing the allele-specific primers. A colour change (pink-mediated by cresol red) confirmed the presence of DNA in the PCR reaction mix.

(iv) PCR Thermal Cycling

PCR was performed using a PTC-200 Peltier Thermal Cycler (MJ Research) The following PCR cycling parameters were used:

5 cycles of:

96° C. for 25 seconds,

70° C. for 45 seconds,

72° C. for 45 seconds;

21 cycles of:

96° C. for 25 seconds,

65° C. for 50 seconds,

72° C. for 45 seconds; and

4 cycles of:

96° C. for 25 seconds,

55° C. for 60 seconds,

72° C. for 125 seconds.

The SSP-PCR products were analysed on a 2 percent agarose gel and visualised by ethidium bromide incorporation. A high-resolution digital photograph was taken and the genotype determined by gene counting.

As a quality assurance measure, genotyping was performed by two independent investigators who were blinded to the origin of DNA. They found 100 percent concordance.

### EXAMPLE 4

Statistical Analysis

Statistical analysis of data derived from the study was performed using Stata statistical software (Version 7.0, College Station, Tex., USA). The baseline characteristics between cases and controls were analysed using the chi-square test for categorical variables and the two-tailed t-test for continuous variables. The strength of association between PON1-107 T/C and the PON1 M54L genotypes and ischemic stroke was estimated by calculating the odds ratio (OR) and 95 percent confidence intervals (Cl). The relative strength of association of known risk factors was determined in a similar fashion. A bi-variate chi-square analysis was used to examine the relation between PON1-107 T/C amd PON1 M54L genotypes and each traditional risk factor. This identified the important confounding variables that were incorporated in to an unconditional logistic regression model, which allowed determination of the risk of ischemic stroke associated with the PON1-107 T/C and M54L polymorphisms. Finally, a multivariate analysis stratified for ischemic stroke subtype was performed. A two tailed p-value of <0.05 was considered significant.

### EXAMPLE 5

Subject Selection

Two hundred and one (201) patients who were admitted with acute ischemic stroke to one of five major hospitals within metropolitan Adelaide, South Australia, were approached to participate in the study of which one hundred and eight two (182) (90.5%) agreed to participate. One hundred and thirty-seven patients (137) (75%) presented with their first ever ischemic stroke.

The diagnosis of ischemic stroke was made by a neurologist in accordance with the World Health Organization (WHO) definition (as described in Hatono, S. (1976) “Experience from a multicentre stroke register: a preliminary report” Bull WHO 54: 541-553). A brain computerized tomography (CT) or magnetic resonance imaging (MRI) was performed on all subjects to exclude intracerebral haemorrhage. Neuroimaging also aided in the confirmation of ischemic stroke and its localisation. Ischemic stroke was further sub-typed using the Oxfordshire Community Stroke Project (OCSP) classification system (as described in Bamford, J., P. Sandercock, M. Dennis, et al. (1991) “Classification and natural history of clinically identifiable subtypes of cerebral infarction” Lancet 337(8756): 1521-1526) into four categories: (a) Total anterior circulation syndrome; (b) Partial anterior circulation syndrome; (c) Posterior circulation syndrome and (d) Lacunar syndrome.

The control group comprised of 301 non-hospitalized subjects who resided in metropolitan Adelaide and did not have a personal history of cerebrovascular disease. Controls were selected via random sampling of the South Australian electronic telephone directory and matched with patients for age (within five- year strata) and gender.

Following informed consent, subjects were interviewed and demographic information was recorded. Cerebrovascular risk factors including hypertension, hypercholesterolemia, and diabetes were considered to be present if there was a reported history or the individual was receiving medical treatment for the condition(s) at the time of investigation. An electrocardiogram was undertaken to determine the presence of atrial fibrillation. Subjects were considered as smokers if they had smoked cigarettes or tobacco on a regular basis within the last five years. A history of stroke in a first degree relative was also recorded. The study was approved by the North. West Adelaide Health Service Ethics Committee.

### EXAMPLE 6

Demographic Characteristics and Prevalence of Risk Factors for Patients with Ischemic Stroke and Healthy Controls

The demographic characteristics and prevalence of risk factors for cerebrovascular disease for 182 ischemic stroke cases and 301 controls are shown in Table 1. As the study matched cases and controls for age and gender, no differences were observed between the two groups for these variables. The study population was predominantly of Caucasian origin with the difference between cases and controls not achieving statistical significance. Of the known risk factors examined, atrial fibrillation was associated with the highest risk of ischemic stroke, with 23% of cases versus 3% of controls affected (OR 8.5, 95% Cl 4.1-17.4). A history of smoking within the last five years (OR 3.1, 95% Cl 1.9-5.2) and diabetes (OR 2.7, 1.6-4.4) were also found to be significantly associated with ischemic stroke. The prevalence of hypertension was higher in cases than controls, however the difference was not statistically significant. No association was observed between ischemic stroke and a history of stroke in a first degree relative or hypercholesterolemia (Table 1).

TABLE 1
Demographic Characteristics and Prevalence of Risk Factors
for Patients with Ischemic Stroke and Healthy Controls

| | Controls | Cases | Odds Ratio | |
|---|---|---|---|---|
| | (n = 301) | (n = 182) | (95% CI) | p value |
| Age* (years) | 73.4 ± 11.6 | 73.6 ± 12 | | 0.8 |
| Gender | | | | |
| Females | 134 (45) | 80 (44) | | |
| Males | 167 (55) | 102 (56) | 1.0 (0.7-1.5) | 0.9 |
| Ethnic Origin | | | | |
| Caucasian | 299 (99.7) | 178 (98) | | |
| Non-Caucasian | 1 (0.3) | 4 (2) | 6.7 (0.8-60.6) | 0.09 |
| Smoking | | | | |
| No | 271 (90) | 135 (74) | | |
| Yes | 30 (10) | 47 (26) | 3.1 (1.9-5.2) | <0.0001 |
| Hypertension | | | | |
| No | 164 (54) | 87 (48) | | |
| Yes | 137 (46) | 85 (52) | 1.3 (0.9-1.9) | 0.2 |
| Diabetes Mellitus | | | | |
| No | 268 (89) | 137 (75) | | |
| Yes | 33 (11) | 45 (25) | 2.7 (1.6-4.4) | <0.0001 |
| Atrial Fibrillation | | | | |
| No | 291 (97) | 141 (77) | | |
| Yes | 10 (3) | 41 (23) | 8.5 (4.1-17.4) | <0.0001 |
| Family History | | | | |
| No | 201 (67) | 122 (67) | | |
| Yes | 100 (33) | 60 (33) | 1.0 (0.7-1.5) | 0.95 |
| Hyper- | | | | |
| cholesterolemia | | | | |
| No | 182 (60) | 117 (64) | | |
| Yes | 119 (40) | 65 (36) | 0.9 (0.6-1.2) | 0.4 |

*Age is expressed as a mean ± standard deviation
In other rows, the values denote the number of patients or controls affected followed by (in parentheses) the percentage of the total for that group

### EXAMPLE 7

Prevalence of the PON1-107 TIC Polymorphism Among Patients with Ischemic Stroke and Healthy Controls

Two stroke patients died prior to venous blood sampling, thus genetic analysis (as described in Example 3) could not be performed in these cases. Table 2 shows the prevalence of the PON1-107 T/C polymorphism in the remaining 180 ischemic stroke cases and 301 controls. Among the control group, 20% were homozygous for the T allele (TT), 66% were heterozygous (TC) and 13% were homozygous for the T allele (TT). The respective genotype distribution in the ischemic stroke cohort was 24% (TT), 54% (TC) and 22% (CC). After adjustment for known risk factors, none of the genotypes was found to be a significant and independent predictor of ischemic stroke.

TABLE 2
Prevalence of the PON1 - 107 T/C polymorphism among patients
with ischemic stroke and healthy controls

| PON1 - | | | | | Multivariate | |
|---|---|---|---|---|---|---|
| 107 T/C | | | Univariate | | Analysis* | |
| Geno- | Controls | Cases | OR | p | OR | p |
| type | n (%) | n (%) | (95% CI) | value | (95% CI) | value |
| TT | 61 (20) | 44 (24) | 1.0 | | 1.0 | |
| TC | 200 (66) | 97 (54) | 0.7 (0.4-1.1) | 0.1 | 0.7 (0.4-1.1) | 0.1 |
| CC | 40 (14) | 39 (22) | 1.4 (0.8-2.4) | 0.3 | 1.4 (0.8-2.4) | 0.3 |

*Multivariate model includes ethnic origin, hypertension, diabetes, hypercholesterolemia, family history for stroke, smoking and atrial fibrillation

### EXAMPLE 8

Distribution of the PON1-107 TIC Genotypes Among Patients Stratified for Stroke Subtype and Controls

The distribution of the PON1-107 T/C genotypes among patients stratified for stroke subtype and controls is shown in Table 3. Forty-four (24.5%) of ischemic stroke patients were classified with lacunar stroke. In this sub-group, the CC homozygous state was significantly associated with lacunar stroke on univariate analysis (OR 2.4, 95% Cl 1.01-5.9, p=0.05). Adjustment for known risk factors using an unconditional logistic regression model did not alter the significance of this finding (Table 3). In contrast, no association between the PON1-107 T/C genotypes and risk of non-lacunar stroke was observed.

TABLE 3
Prevalence of the PON1 - 107 polymorphism among patients with ischemic stroke
stratified for stroke subtype and healthy controls

| PON1 - 107 | | Lacunar Stroke | | | | | |
|---|---|---|---|---|---|---|---|
| C/T | Control | | | p | Non-Lacunar Stroke* | | |
| Genotype | n (%) | n (%) | OR (95% CI) | value | n (%) | OR (95% CI) | p value |
| TT | 61 (20) | 10 (23) | 1.0 | | 34 (25) | 1.0 | |
| TC | 200 (66) | 18 (41) | 0.6 (0.2-1.3) | 0.2 | 79 (58) | 0.7 (0.4-1.2) | 0.2 |
| CC | 40 (13) | 16 (36) | 2.4 (1.01-5.9) | 0.05 | 23 (17) | 1.0 (0.5-2.0) | 0.9 |

*Non-lacunar stroke defined as stroke patients presenting with either total anterior circulation syndrome, partial anterior circulation syndrome and posterior circulation syndrome. Odds ratios were determined by multivariate analysis that included ethnic origin, hypertension, diabetes, hypercholesterolemia, family history for stroke, smoking and atrial fibrillation.

### EXAMPLE 9

Prevalence of the PON1 M54L Polymorphism Among Patients with Ischemic Stroke and Healthy Controls

The PON1 gene also has a SNP resulting in a methionine (M) to Leucine (L) substitution at amino acid position 54.

Two stroke patients died prior to venous blood sampling, thus genetic analysis (as described in Example 3) could not be performed in these cases.

Table 4 shows the prevalence of the PON1 M54L polymorphism in the 180 ischemic stroke cases and 301 controls. Among the control group, 12% were homozygous for the MM allele, 46% were heterozygous (ML) and 42% were homozygous for the LL allele. The respective genotype distribution in the ischemic stroke cohort was 11% (MM), 46% (ML) and 43% (LL). After adjustment for known risk factors, none of the M54L genotypes was found to be a significant and independent predictor of ischemic stroke.

TABLE 4
Prevalence of the PON1 M54L polymorphism among patients
with ischemic stroke and healthy controls

| PON1 | | | | | Multivariate | |
|---|---|---|---|---|---|---|
| M54L | | | Univariate | | Analysis* | |
| Geno- | Controls | Cases | OR | p | OR | p |
| type | n (%) | n (%) | (95% CI) | value | (95% CI) | value |
| MM | 36 (12) | 20 (11) | 1.0 | | 1.0 | |
| ML | 138 (46) | 82 (46) | 1.1 (0.6-2.0) | 0.8 | 1.2 (0.6-2.4) | 0.6 |
| LL | 127 (42) | 78 (43) | 1.1 (0.6-2.0) | 0.7 | 1.0 (0.5-2.1) | 1.0 |

*Multivariate model includes ethnic origin, hypertension, diabetes, hypercholesterolemia, family history for stroke, smoking and atrial fibrillation

### EXAMPLE 10

Distribution of the PON1 M54L Genotypes Among Patients Stratified for Stroke Subtype and Controls

The distribution of the PON1 M54L genotypes among patients stratified for stroke subtype and controls is shown in Table 5. Forty-four (24.5%) of ischemic stroke patients were classified with lacunar stroke. In this sub-group, none of the M54L genotypes was significantly associated with lacunar stroke on univariate analysis. Adjustment for known risk factors using an unconditional logistic regression model did not alter the significance of this finding (Table 5).

TABLE 5
Prevalence of the PON1 M54L polymorphism among patients with ischemic stroke
stratified for stroke subtype and healthy controls

| | | Lacunar Stroke | | | | | |
|---|---|---|---|---|---|---|---|
| PON1 M54L | Control | | | p | Non-Lacunar Stroke* | | |
| Genotype | n (%) | n (%) | OR (95% CI) | value | n (%) | OR (95% CI) | p value |
| MM | 36 (12) | 3 (7) | 1.0 | | 17 (13) | 1.0 | |
| ML | 138 (46) | 25 (57) | 2.2 (0.6-7.6) | 0.2 | 57 (42) | 0.9 (0.5-1.7) | 0.7 |
| LL | 127 (42) | 16 (36) | 1.5 (0.4-5.5) | 0.5 | 62 (46) | 1.0 (0.5-2.0) | 0.9 |

*Non-lacunar stroke defined as stroke patients presenting with either total anterior circulation syndrome, partial anterior circulation syndrome and posterior circulation syndrome. Odds ratios were determined by multivariate analysis that included ethnic origin, hypertension, diabetes, hypercholesterolemia, family history for stroke, smoking and atrial fibrillation.

### EXAMPLE 11

PON1 Enzymatic Activity

PON1 enzymatic activity may be determined by measurement in human serum samples, using either phenylacetate (arylesterase activity) or paraoxan (paraoxonase activity) as substrates.

For example, PON1 activity may be determined in human serum as described in Furlong et al. (1993) Chem Biol Interact 87:35-48 or Blattner Garin et al. (1994). Biochem J. 304:549-554.

### EXAMPLE 12

Cell Culture

Hep G2 cells may be obtained from the ATCC.

Medium: Minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10%. Temperature: 37.0 C

For subculturing, the culture medium may be removed and discarded. The cell layer may then be briefly rinsed with 0.25% (w/v) Trypsin-0.53 mM EDTA solution to remove all traces of serum that contains trypsin inhibitor. Then 2.0 to 3.0 ml of Trypsin-EDTA solution may be added to the flask and cells observed under an inverted microscope until cell layer is dispersed (usually within 5 to 15 minutes). To avoid clumping it is preferable to not agitate the cells by hitting or shaking the flask while waiting for the cells to detach. Cells that are difficult to detach may be placed at 37° C. to facilitate dispersal. 6.0 to 8.0 ml of complete growth medium may be added and cells aspirated by gently pipetting. An appropriate aliquot of the cell suspension may then be added to new culture vessels. Incubate cultures at 37° C. A subcultivation ratio of 1:4 to 1:6 may be used with a twice weekly renewal.

### EXAMPLE 13

Transfection of HepG2 Cells with PON1 Upstream Region

A DNA fragment containing a 1027 bp DNA fragment of the PON1 upstream region having either the -107 T allele sequence or the polymorphic -107 C allele may be derived from the appropriate human genomic clones or by PCR amplification from genomic DNA, essentially as described in Leviev et al. (2000) Arterioscler Thromb Vasc Biol 20(2):516-521.

The DNA may then be fused to the chloramphenicol acetyltransferase (CAT) reporter gene. Transfection of HepG2 cells with the PON1 promoter-CAT gene constructs may be performed by employing the calcium phosphate precipitation method, incorporating a glycerol shock, as described in Sambrook, J, Fritsch, E. F. and Maniatis, T. Molecular Cloning: A Laboratory Manual 2nd. ed. Cold Spring Harbor Laboratory Press, New York. (1989).

After transfection, cells may be harvested and relative changes in CAT activity quantitated by determining the percentage of [¹⁴C]chloramphenicol converted to its acetylated products by thin-layer chromatography and liquid scintillation counting, as described in Sambrook, J, Fritsch, E. F. and Maniatis, T. Molecular Cloning: A Laboratory Manual 2nd. ed. Cold Spring Harbor Laboratory Press, New York. (1989).

The transfection efficiency may be evaluated by cotransfecting cells with cytomegalovirus promoter and lac Z gene construct. β-Galactosidase assay may be performed as described in Sambrook, J, Fritsch, E. F. and Maniatis, T. Molecular Cloning: A Laboratory Manual 2nd. ed. Cold Spring Harbor Laboratory Press, New York. (1989), and may be taken as a direct index of the efficiency of transfection and used to normalize CAT activities among various experiments.

Finally, it will be appreciated that various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the fields of vascular biology, neurology, molecular biology or related fields are intended to be within the scope of the present invention.

1-56. (canceled)

57. A method of identifying a subject predisposed to small vessel occlusion, wherein said method comprises: identifying the presence of a thymine to cytosine mutation at position -107 in both alleles of the paraoxonase 1 locus, wherein the presence of the mutation in both alleles indicates the subject is predisposed to small vessel occlusion.

58. The method according to claim 57, wherein the small vessel occlusion is a lacunar stroke, cerebral leukoaraiosis, dementia, ischemic heart disease including ischemic cardiomyopathy, peripheral vascular disease, disseminated intravascular coagulation, small vessel vasculitis, ischemic neuropathy, ischemic retinopathy, ischemic gastropathy including small and large bowel ischemia, diffuse pulmonary embolism, and vascular impotence.

59. The method according to claim 57, wherein the small vessel occlusion occurs in the brain.

60. The method according to claim 57, wherein the identification of the mutation comprises amplification of a region containing the mutation from nucleic acid isolated or derived from the subject.

61. The method according to claim 57, wherein the identification of the mutation comprises detection of the mutation by hybridisation of nucleic acid isolated or derived from the subject to a reporter nucleic acid.

62. The method according to claim 57, wherein the subject predisposed to small vessel occlusion is predisposed to developing a disease or condition associated with small vessel occlusion.

63. The method according to claim 57, wherein the subject is suitable for treatment with an agent that decreases the activity of paraoxonase 1.

64. A method of determining the risk of small vessel occlusion in a subject, wherein said method comprises: identifying the presence of a thymine to cytosine mutation at position -107 in one or both alleles of the paraoxonase 1 locus, wherein the presence of the mutation in both alleles indicates an increased risk that the subject is predisposed to small vessel occlusion.

65. The method according to claim 64, wherein the risk of small vessel occlusion is compared to the risk of small vessel occlusion for a subject in the general population.

66. The method according to claim 64, wherein the small vessel occlusion is a lacunar stroke, cerebral leukoaraiosis, dementia, ischemic heart disease comprising ischemic cardiomyopathy, peripheral vascular disease, disseminated intravascular coagulation, small vessel vasculitis, ischemic neuropathy, ischemic retinopathy, ischemic gastropathy including small and large bowel ischemia, diffuse pulmonary embolism, and vascular impotence.

67. The method according to claim 64, wherein the small vessel occlusion occurs in the brain.

68. The method according to claim 64, wherein the identification of the mutation comprises amplification of a region containing the mutation from nucleic acid isolated or derived from the subject.

69. The method according to claim 64, wherein the identification of the mutation comprises detection of the mutation by hybridisation of nucleic acid isolated or derived from the subject to a reporter nucleic acid.

70. A method of preventing and/or treating a subject susceptible to a small vessel occlusion, the method including the step of administering to the subject an effective amount of an agent that decreases the activity of paraoxonase 1.

71. The method according to claim 70, wherein the small vessel occlusion is a small vessel occlusion in the brain.

72. The method according to claim 70, wherein the small vessel occlusion is a lacunar stroke, cerebral leukoaraiosis, dementia, ischemic heart disease including ischemic cardiomyopathy, peripheral vascular disease, disseminated intravascular coagulation, small vessel vasculitis, ischemic neuropathy, ischemic retinopathy, ischemic gastropathy including small and large bowel ischemia, diffuse pulmonary embolism, or vascular impotence.

73. An isolated nucleic acid that (i) consists of the sequence according to SEQ. ID No.3 or SEQ ID NO.4, or a RNA equivalent thereof; or (ii) includes one or more base substitutions in the sequence according to SEQ ID No. 3 or SEQ ID NO.4, wherein the nucleic acid with one or more base substitutions has at least 80% homology to SEQ. ID No.3 or a RNA equivalent thereof; or (iii) includes one or more base substitutions in the sequence according to SEQ ID No. 3 or SEQ ID NO.4, wherein the nucleic acid with one or more base substitutions hybridises with the complement of SEQ ID No.3 under stringent hybridisation conditions and the stringent hybridisation conditions include hybridisation in 6×SSC at 42° C. and washing in 2×SSC at 20° C.
